(19) United States
(12) Patent Application Publication
(10) Pub. No.: US 2016/0158429 A1
(43) Pub. Date: Jun. 9, 2016

(54) HYPERTHERMIC BODY FLUID TREATMENT SYSTEM AND METHOD FOR THE SAME

(71) Applicant: White Oak Industries Inc., Archdale, NC (US)

(72) Inventor: Anthony T. Harrelson, Sophia, NC (US)

(21) Appl. No.: 14/632,440

(22) Filed: Feb. 26, 2015

Related U.S. Application Data
(60) Provisional application No. 62/089,661, filed on Dec. 9, 2014.

(51) Int. Cl. A61M 1/36; A61M 1/16

(52) U.S. Cl. CPC A61M 1/369; A61M 1/3609; A61M 1/1603

(57) ABSTRACT

A method for hyperthermic treatment of body fluid includes diverting at least a portion of a body fluid through a body fluid inlet from a body. A viral load of a pathogen is measured in the body fluid, and a target treatment temperature is determined based on the measured viral load. The body fluid is heated to the target treatment temperature to decrease the viral load in the body fluid. The body fluid is returned to the body after hyperthermic treating through a body fluid outlet. Hyperthermic treating of the body fluid is repeated with another portion of body fluid diverted through the hyperthermic treatment assembly.

### CROSS-REFERENCE TO RELATED PATENT DOCUMENTS

This patent application claims priority under 35 USC §119(e) to U.S. Provisional Patent Application Ser. No. 62/089,661, filed on Dec. 9, 2014; entitled HYPERTHERMIC BLOOD TREATMENT SYSTEM AND METHOD FOR THE SAME which is incorporated by reference herein.

### TECHNICAL FIELD

This document pertains generally, but not by way of limitation, to heat transfer with body fluids as a form of treatment for chronic infectious diseases.

### BACKGROUND

Immunotherapy, also called biologic therapy or biotherapy, is the treatment of disease by inducing, enhancing, or suppressing an immune response. In other words, immunotherapy is treatment that uses certain parts of a patient's immune system to fight diseases. Immunotherapies designed to elicit or amplify an immune response are classified as activation immunotherapies. This can be done in a couple of ways, for example, by stimulating a patient's own immune system to work harder or smarter to attack disease or by giving the immune system components, such as man-made immune system proteins (materials either made by the body or in a laboratory to improve, target, or restore immune system function). Immunotherapies that reduce or suppress are classified as suppression immunotherapies.

The active agents of immunotherapy are collectively called immunomodulators. They are a diverse array of recombinant, synthetic and natural preparations, including by not limited to cytokines, antibodies, vaccines, non-specific immunotherapies, granulocyte colony-stimulating factor (G-CSF), interferons, imiquimod, cellular membrane fractions from bacteria, IL-2, IL-7, IL-12, various chemokines, synthetic cytosine phosphate-guanosine (CpG) oligodeoxynucleotides and glucans. Immunomodulatory regimens offer an attractive approach as they often have fewer side effects than existing drugs, including less potential for creating resistance in microbial diseases.

### Overview

The present inventors have recognized, among other things, that a problem to be solved can include improving the quality of treatment of a pathogen beyond that provided by pharmacological intervention, such as immunotherapy (e.g., application of medicaments). The present inventor has recognized, among other things, that a problem to be solved can include providing a supplemental or standalone form of treatment including hyperthermic treatment (heating) of blood carrying a pathogen and pathogen infected cells. Such pathogens include, but are not limited to, Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus or HIV.

As discussed herein, in one example a hyperthermic treatment system diverts blood flow from the affected patient for instance by providing communication across the femoral arteries. The inflow of blood from one of the femoral arteries is moved through the system by a pump, for instance a peristaltic pump (e.g., a roller pump) or the like. The inflowing blood is delivered through a heat exchanger and correspondingly heated to a target treatment temperature configured to trigger pathogen and infected cell death. The target treatment temperature is determined based on the measurement of the present viral load of the pathogen of interest in the blood and associating the measurement with a corresponding treatment temperature (e.g., found in a standalone table, database for a controller or the like). In one example, higher temperatures are used with higher viral loads and relatively lower temperatures are used with lower viral loads. The blood is heated to the target temperature and pathogen infected cells and the pathogen suffer cell death and are captured in a filter. The filtered and heated blood is then cooled, for instance actively cooled with a second heat exchanger, to a temperature near body temperature, prior to returning the blood to the patient (e.g., to the second femoral artery).

The system and method described above enhances the effectiveness of immunotherapy treatments. For instance, by treating the patient with medicaments or the like beforehand the viral load of the patient is decreased prior to use of the hyperthermic treatment system. The hyperthermic treatment system provides a second treatment mechanism that further decreases the viral load of the patient. In another example, hyperthermic treatment is conducted as a first treatment mechanism and immunotherapy is used as a second treatment. In still another example, hyperthermic treatment is used as a standalone treatment to decrease viral load.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic view of a hyperthermic treatment system.

FIG. 2 is a schematic view of one example of a hyperthermic treatment system including at least a hyperthermic heat exchanger.

FIG. 3 is a perspective view of one example of a hyperthermic heat exchanger configured to decrease viral load in a body fluid.

FIG. 4A is a cross-sectional view of one example of a perfusate filter configured to filter dead pathogen cells and dead pathogen infected cells for the body fluid.

FIG. 4B is a cross-sectional view of another example of a perfusate filter configured to filter dead pathogen cells and dead pathogen infected cells for the body fluid.

FIG. 5 is a perspective view of one example of a cooling heat exchanger configured to cool the body fluid before return to a body.

FIG. 6 is a block diagram showing one example of hyperthermic treatment of a body fluid t no decrease viral load.

FIG. 7 is a block diagram showing another example of hyperthermic treatment of a body fluid to decrease viral load.

### DETAILED DESCRIPTION

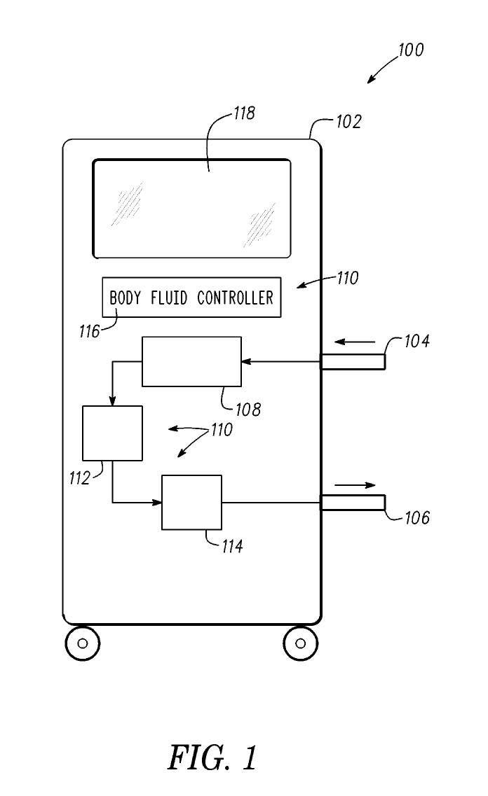

FIG. 1 shows one example of hyperthermic treatment system 100. The example hyperthermic treatment system 100 includes a system housing 102 including the components of a hyperthermic treatment assembly 110 therein. The hyperthermic treatment system 100 diverts a body fluid, such as blood, therethrough and treats the body fluid for one or more pathogens within the body fluid. For instance, as shown in FIG. 1 a body fluid inlet 104 (e.g., configured for communication with a femoral artery) extends into the system housing 102 into a circuit of components including components of the hyperthermic treatment assembly 110. After treatment of the body fluid the body fluid is returned to the body, for instance to an opposed femoral artery, through a body fluid outlet 106. The hyperthermic treatment system 100 decreases the viral load (quantity of pathogen) of the body fluid through the controlled application of heat to the body fluid to trigger cell death of the pathogen infected cells. In one example, and as described herein the hyperthermic treatment assembly 110 heats the body fluid to a target treatment temperature (based on the detected viral load of the body fluid) to trigger the death of the pathogen and pathogen infected cells in the body fluid. In another example, the body fluid is cooled to a return temperature after treatment, for instance with a cooling heat exchanger prior to return of the body fluid through the body fluid outlet 106.

The hyperthermic treatment system 100 and hyperthermic treatment assembly 110 are used alone or in combination with another form of immunotherapy (e.g., medicaments or the like) to decrease the viral load of a pathogen in the body fluid, such as the blood of an animal. Pathogens treated with the hyperthermic treatment system 100 and the hyperthermic treatment assembly 110 include, but are not limited to, Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, and HIV. The system 100 and assembly 110 are not limited to these pathogens and are instead applicable with any pathogen that suffers cell death with the elevation of body fluid temperature.

As shown in FIG. 1, the hyperthermic treatment system 100 includes a circuit of components within the system housing 102. The body fluid inlet 104 and body fluid outlet 106 in the hyperthermic treatment system 100 are configured to divert a flow of the body fluid from an animal body (animal as used herein includes any sort of animal including humans, mammals, reptiles, other non-sentient animals or the like). The body fluid is delivered through the body fluid inlet 104 and, after treatment by the hyperthermic treatment assembly 110, is returned to the body, for instance through the body fluid outlet 106. A circuit through the system housing 102 is configured to pump the body fluid therethrough and heat the body fluid with a hyperthermic heat exchanger to trigger the death of pathogen cells or pathogen infected cells in the body fluid prior to return through the body fluid outlet 106. The process performed by the hyperthermic treatment system 100 is optionally repeated in the manner of a bypass system that continuously or near continuously cycles the body fluid from the body through the system 100 and then returns the body fluid to the animal. Stated another way, the body fluid is continuously cycled from the body through the hyperthermic treatment system 100 according to a specified treatment time, detected instant viral load and viral load target or the like.

Referring again to FIG. 1, the hyperthermic treatment system 100 includes an input/output device 118 provided on the system housing 102. In one example, the input/output device 118 includes one or more diagnostic features for instance blood pressure monitors, flow rate monitors, oxygen content monitors (of the body fluid), temperature of the body fluid at various points in the hyperthermic treatment system 100 circuit and the like. In another example, the input/output device 118 includes one or more input features including, but not limited to, a touchscreen interface, keyboard, key pad or the like configured to input one or more values to the hyperthermic treatment system 100. For instance the input/output device 118 is used to input a measured viral load, pathogen type, animal (patient) information or the like into the system 100 for instance for use by a body fluid controller 116.

As described herein the viral load input of a particular pathogen to the input/output device 118 is used to determine a corresponding target treatment temperature for use with the hyperthermic heat exchanger 112. That is to say, the body fluid controller 116 optionally includes a database of a plurality of pathogens and viral load and target treatment temperature combinations for each of the pathogens. The body fluid controller, in an example, automatically determines a target treatment temperature based on the input viral load in combination with the identification of the pathogen to be treated. In still another example, the input/output device 118 is used to manually enter a target treatment temperature for use with the hyperthermic treatment assembly 110 to accordingly treat a pathogen.

The body fluid controller 116 shown in the system housing 102 is in communication with the input/output device 118 as well as a number of features within the hyperthermic treatment assembly 110 including the hyperthermic heat exchanger 112 and an optional cooling heat exchanger 114. The body fluid controller 116 receives one or more of a determined viral load and pathogen of an animal coupled with the body fluid inlet and outlet 104, 106. After inputting one or more of the pathogen as well as the animal viral load the body fluid controller 116 (e.g., using a database of known pathogens and viral load and target treatment temperature pairings) delivers a target treatment temperature to the hyperthermic heat exchanger 112 and controls the hyperthermic heat exchanger 112 to raise the temperature of the body fluid to the specified target treatment temperature. As discussed herein, the target treatment temperature triggers the death of a pathogen or pathogen infected cells in the body fluid while the body fluid is passed through the hyperthermic treatment system 100. In another example, the body fluid controller 116 cooperates with a plurality of temperature sensors, for instance in the circuit including the hyperthermic treatment assembly 110, to measure the temperature of the body fluid passing through the hyperthermic heat exchanger 112. The body fluid controller uses the temperature measures to control the hyperthermic heat exchanger 112 (and optionally the cooling heat exchanger 114) by way of feedback control to achieve the target treatment temperature. In a further example, the body fluid controller 116 uses temperature sensors inline in the circuit shown in FIG. 1 to measure the input and output temperatures of the body fluid flowing into and through the cooling heat exchanger 114 and accordingly controls the cooling heat exchanger 114 to achieve a return body fluid temperature similar to the body temperature of the animal coupled with the hyperthermic treatment system 100. That is to say, the body fluid controller 116 in an example uses a plurality of inline temperature sensors in the flow circuit in the system 100 and at one or more the inputs or outputs of features of the hyperthermic treatment assembly 110 (e.g., the hyperthermic heat exchanger 112 and the cooling heat exchanger 114) to accordingly raise and lower the body fluid temperature to achieve both death of the pathogen and pathogen infected cells and conditioning of the body fluid to a temperature nearer to the body fluid temperature prior to return to the body of the animal (through the body fluid outlet 106).

As further shown in FIG. 1, in one example the circuit of features within the hyperthermic treatment system 100 include a body fluid pump 108. The body fluid pump 108 provides the flow of body fluid from the body fluid inlet 104 into the body fluid outlet 106. The body fluid pump 108 is optionally in communication with the body fluid controller 116 (for instance it is controlled by the body fluid controller 116) to provide a target flow rate of the body fluid to each of the features of the hyperthermic treatment assembly 110 including one or more of the hyperthermic heat exchanger 112 and the cooling heat exchanger 114. That is to say, in another example the body fluid controller 116 controls both the flow rate and the temperature of the body fluid through the hyperthermic treatment system 100 to accordingly reach a desired target treatment temperature at the hyperthermic heat exchanger 112, a return temperature at the cooling heat exchanger 114, and to do so at a desired flow rate for instance a flow rate matching a body fluid removal flow rate through the body fluid inlet 104 and a desired body fluid return flow rate at the body fluid outlet 106.

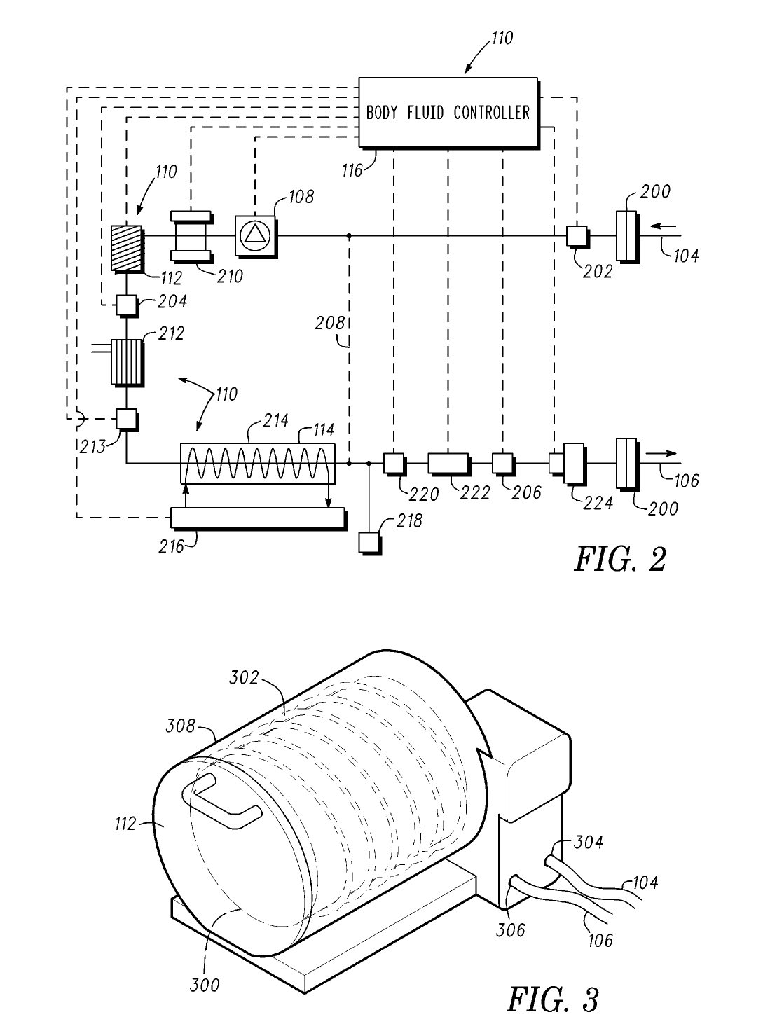

FIG. 2 shows a detailed schematic view of the example hyperthermic treatment assembly 110. In FIG. 2 the hyperthermic treatment assembly 110 is an inline series of components of an overall fluid flow circuit through the hyperthermic treatment system 100. The fluid flow circuit includes the body fluid inlet 104 (and body fluid out let 106) and one or more flow clamps 200. The body fluid inlet and outlet 104, 106 included one or more of ports, peripheral catheters or the like or interfaces configured to couple with the same to provide fluid communication between the body of an animal and the hyperthermic treatment system 100. The flow clamps 200 in one example provide a mechanical feature that engages with the body fluid inlet 104 (for instance a pliable tube) to close flow into the flow circuit of the hyperthermic treatment system 100.

Continuing along the flow circuit, a first temperature sensor 202 is provided after the body fluid inlet 104 and prior to the body fluid pump 108. In one example, the first temperature sensor 202 is positioned elsewhere within the flow circuit, for instance immediately prior to the hyperthermic heat exchanger 112. The first temperature sensor 202 cooperates with a second temperature sensor 204 downstream from the sensor 202 in another example to accordingly measure the input and output temperatures for the hyperthermic heat exchanger 112. As discussed herein, the input and output temperature measurements used by the body fluid controller 116 to adjustment the heating of the hyperthermic heat exchanger 112.

Optionally, a bypass line 208 is provided across the flow circuit of the hyperthermic treatment system 200 to accordingly provide an emergency bypass around the components of the hyperthermic treatment system. The bypass line 208 facilitates direct or near direct communication between the body fluid inlet 104 and the body fluid outlet 106.

As further shown in FIG. 2, the body fluid pump 108 is provided in the flow circuit of the hyperthermic treatment system 100. In the example shown in FIG. 2 the body fluid pump 108 is a rotary pump that provides a measured and controlled flow rate of the body fluid through the hyperthermic treatment system 100. In another example, the body fluid pump 108 includes any sort of metering pump configured to provide a precise flow of a body fluid through the system. For instance the body fluid pump 108 includes, but is not limited to, a rotary pump, peristaltic pump or the like.

In another example, a gas bubble sensor 210 is provided downstream from the body fluid pump 108. Optionally, the gas bubble sensor 210 is positioned in other portions of the flow circuit of the hyperthermic treatment system 100. For instance, the gas bubble sensor 210 is positioned downstream from any feature that may generate gas bubbles within the flow of the body fluid prior to the return to the body (e.g., through the body fluid outlet 106). In one example, the gas bubble sensor 210 includes one or a plurality of different sensor types configured to detect gas bubbles such as air bubbles within the body fluid flow. For instance, the gas bubble sensor includes, but is not limited to, an infrared sensor, a reflective light sensor, an ultrasound sensor or the like.

Referring again to FIG. 2, the hyperthermic heat exchanger 112 is provided in the flow circuit of the hyperthermic treatment system 100. In one example, the hyperthermic heat exchanger 112 includes a heating core and a body fluid conduit. In one example, the body fluid conduit wraps around the heating core to facilitate heat exchange between the heating core and the body fluid within the body fluid conduit. The hyperthermic heat exchanger 112 as described herein raises the temperature of the body fluid to a target treatment temperature configured to trigger the death of a pathogen or pathogen infected cells in the body fluid.

As will be further described herein, in one example the first temperature sensor 202 and the second temperature sensor 204 (corresponding to input and output temperature sensors for the hyperthermic heating exchanger 112) are used in combination by the body fluid controller 116 to automatically control the temperature of the hyperthermic heat exchanger 112. That is to say, the body fluid controller 116 achieves the target treatment temperature by measuring the first and second temperatures at the first and second temperature sensors 202, 204 and accordingly adjusts the heat applied from the heating core to the body fluid in the body fluid conduit to ensure elevation of the body fluid.

As further shown in FIG. 2, the hyperthermic treatment assembly 110 includes a filter 212. In one example, the filter 212 is an inline perfusate filter provided between the hyperthermic heat exchanger 112 and the cooling heat exchanger 114. As described further herein, the perfusate filter 212 includes a plurality of perforated flow channels or tubes. The perforated flow channels are exposed to a crossing flow of infusion fluid such as saline or the like that is delivered into a waste removal reservoir provided around the perforated flow channels. The saline entrains dead or dying pathogen cells and pathogen infected cells from the perforations. The entrained wastes are drawn with the infusion fluid from the filter 212. The remainder of the body fluid including healthy blood cells, white blood cells or the moves downstream towards the body fluid outlet 106 and other components of the hyperthermic treatment system 100.

As further shown in FIG. 2 the hyperthermic treatment assembly 110 includes an optional cooling heat exchanger 114. As discussed herein the hyperthermic heat exchanger 112 raises the body fluid temperature to a target treatment temperature. The cooling heat exchanger lowers the elevated body fluid temperature to avoid shocking the system of the animal. The cooling heat exchanger 114 provides active heat exchange to the body fluid prior to its reintroduction through the body fluid outlet 106. As shown in FIG. 2, the cooling heat exchanger 114 includes a heat exchange jacket 214 and a coolant conditioner 216. In one example the coolant conditioner 216 provides chilled water (or other chilled refrigerant) to the heat exchange jacket 214 (e.g., through a cooling coil within the heat exchange jacket 214) to cool the body fluid flowing through a center passage toward the body fluid outlet 106.

Optionally, the second temperature sensor 204 and a third temperature sensor 206 are used in combination by the body fluid controller 116 to measure the input and output temperatures of the body fluid into and out of the cooling heat exchanger 114 and facilitate the accurate control and cooling of the body fluid to a desired temperature. In one example, the body fluid controller 116 uses the measured input and output temperatures from the sensors 204, 206 to control the heat transfer at the cooling heat exchanger 114 and lower the body fluid temperature to a temperature equivalent to the body temperature of the animal coupled with the hyperthermic treatment system 100. Optionally, the cooling heat exchanger 114 is used to lower the body fluid temperature to a temperature just above the body temperature of the animal to allow for continued heat transfer from the body fluid to the environment (cooling) and reintroduction of the body fluid at the body temperature at the body fluid outlet 106.

As further shown in FIG. 2, in one example the hyperthermic treatment system 100 includes a fluid port 218 (e.g., hemostatis valve, touhy port, piercable diaphragm or the like).

The fluid port 218 provides an injectable port for the application of immunotherapy, medicaments or the like to the flow of the body fluid through the hyperthermic treatment system 100. For instance, a drip line or dedicated immunotherapy injection system is provided at the fluid port 218. That is to say, in one example the hyperthermic treatment system 100 is used to administer one or more medicines or the like to the body fluid in addition to conditioning the body fluid with heat to a target treatment temperature. Optionally, the fluid port 218 facilitates the drawing of body fluid (e.g., by syringe) for diagnostic purposes including updating the measurement of the viral load of an animal coupled to the hyperthermic treatment system 100.

As further shown in FIG. 2 a flow sensor 220 and a flow controller 222 are provided inline with the flow circuit. In one example, the flow sensor 220 measures the flow of the body fluid through the hyperthermic treatment system 100 and cooperates with the flow controller 222 (a valve) to adjust the flow of the body fluid prior to return delivery through the body fluid outlet 106. The body fluid controller 116 is in communication with each of the flow sensor 220 and the flow controller 222 and monitors the flow of the body fluid through the hyperthermic treatment system 100 and ensures precise delivery of the body fluid to the animal with the flow controller 222. For instance, the body fluid controller 116 uses the flow sensor 220 and the flow controller 222 to match the flow rate of the body fluid at the body fluid outlet 106 to the flow rate through the body fluid inlet 104. Accordingly, the body fluid controller 116 in combination with the flow sensor 220 and the flow controller 222 monitors the flow rate of the body fluid through the hyperthermic treatment system 100 and ensures that the inflow of body fluid into the treatment system 100 is identical to or closely matches the outflow of the body fluid to the animal. In still another example, the body fluid pump 108 is used with the flow sensor 220 to control the flow rates into and out of the hyperthermic treatment system 100. Optionally, the body fluid pump and the flow controller 222 are used together to adjust the flow of the body fluid.

Referring again to FIG. 2, a body fluid warmer is optionally provided at the end of the flow circuit. The body fluid warmer 224 provides a heat exchanger immediately prior to the reintroduction of the body fluid to the animal. In one example, the body fluid warmer 224 works in cooperation with the third temperature sensor 206 (in communication with the body fluid controller 116) to adjust the heat transfer to and temperature of the body fluid immediately prior to exit at the body fluid outlet 106. That is to say, in one example the third temperature sensor 206 is also provided immediately adjacent to the body fluid warmer 224 near to the point of reintroduction of the body fluid to the patient, for instance through the body fluid outlet 106. The body fluid warmer 224 provides a final heat exchanger to ensure the returning body fluid is provided to the animal at a desired return temperature (e.g., closely matching the body temperature of the animal). In another example, the body fluid warmer 224 is optional. Stated another way, the hyperthermic heat exchanger 112 and the cooling heat exchanger 114 are operated in cooperation with the body fluid controller 116 and the plurality of temperature sensors (e.g., sensors 202, 204, 206) to both raise and lower the body fluid temperature to a respective target treatment temperature and target return temperature (e.g., body temperature).

Referring again to FIG. 2, the hyperthermic treatment assembly 110 includes the body fluid controller 110, the body fluid pump 108, the hyperthermic heat exchanger 112, the cooling heat exchanger 114 and the filter 212. As previously described herein, the body fluid pump 108 provides a measured flow of body fluid from the body fluid inlet 104 (coupled with a femoral artery) through the hyperthermic treatment assembly 110. The flow of body fluid is delivered through the hyperthermic heat exchanger 112 and accordingly raised to a target treatment temperature. In one example, the target treatment temperature is determined by assessing the viral load of the animal coupled with the hyperthermic treatment system 100. Upon a determination of the viral load as well as identification of the particular pathogen infecting the animal a target treatment temperature is determined, for instance by inputting the target treatment temperature or viral load and pathogen identifier to the body fluid controller 116 (e.g., through the input/output device 118 shown in FIG. 1).

In one example, the hyperthermic heat exchanger 112 in cooperation with a first upstream temperature sensor 202 and a second downstream temperature sensor 204, raises the temperature of the body fluid to the target treatment temperature. For instance, the body fluid controller 116 uses the input and output temperatures at the first and second temperature sensors 202, 204 to accordingly operate the hyperthermic heat exchanger 112 by heating a heating core of the heat exchanger to accordingly raise the temperature of the body fluid within the body fluid conduit of the hyperthermic heat exchanger.

After heating of the body fluid to the desired target treatment temperature the body fluid is delivered within the hyperthermic treatment assembly 110 to the filter 212. As previously described herein, in one example the filter 212 includes a plurality of perforated flow channels surrounded by a waste removal reservoir. A flow of liquid, for instance saline, is provided through the waste removal reservoir and interacts with the perforations of the perforated flow channels to entrain dead and dying pathogen cells and pathogen infected cells out of the flow of the body fluid. The remainder of the clean body fluid (e.g., healthy blood cells, white blood cells and the like) flows from the filter 212 toward the body fluid outlet 106.

In another example, the hyperthermic treatment assembly 110 includes a cooling heat exchanger 114 provided downstream from the filter 212 and upstream from the body fluid outlet 106. The flow of heated body fluid through the cooling heat exchanger 114 is conditioned by the cooling heat exchanger 114 to a temperature substantially equivalent to (matching or nearly matching) the body temperature of the animal coupled with the hyperthermic treatment system 100. For instance, the cooling conditioner 216 provides a flow of cool liquid (chilled water or the like) through a cooling coil within the heat exchange jacket 214. The flow of chilled water through the heat exchange jacket 214 cools the liquid within the heat exchange jacket 214 surrounding a center passage of the cooling heat exchanger 114 having the body fluid therein. The heat exchange jacket 214 and the cooling coil therein cool the body fluid to a desired return temperature. In one example, the second temperature sensor 204 and the third temperature sensor 206 are used as input and output temperature sensors for the cooling heat exchanger 114 by the body fluid controller 116. The body fluid controller 116 uses the input and output temperatures measured with the sensors 204, 206 to control cooling of the body fluid to a temperature matching or nearly matching that of the body prior to delivery through the body fluid outlet 106. As described herein, an optional body fluid warmer 224 is provided downstream within the hyperthermic treatment assembly 110 to ensure delivery of the body fluid to the animal at a desired return temperature prior to delivery through the body fluid outlet 106.

FIG. 3 shows one example of hyperthermic heat exchanger 112 for use with the hyperthermic treatment system 100 shown in FIGS. 1 and 2. In one example, the hyperthermic heat exchanger 112 includes a heating core 300 provided within a body fluid conduit 302. As shown in FIG. 3, the heating core 300 is provided centrally relative to the body fluid conduit 302, and the conduit is wrapped around the heating core 300 in a plurality of circuits in the manner of a coil. As further shown in FIG. 3, the hyperthermic heat exchanger 112 includes input and output ports 304, 306. The input port 304 is in communication with the body fluid inlet 104 (upstream) and the output port 306 is in communication with the body fluid outlet 106 (downstream). The components of the hyperthermic heat exchanger 112 are in one example housed within a heat exchanger body 308. Optionally, the heat exchanger body 308 and the other components of the hyperthermic heat exchanger 112 are housed within the system housing 102 shown in FIG. 1 for the hyperthermic treatment system 100.

As previously described herein the hyperthermic heat exchanger 112 is an inline component of a hyperthermic treatment assembly 110. The hyperthermic heat exchanger 112 raises the temperature of a body fluid, such as blood, to trigger death of a pathogen and pathogen infected cells of the body fluid. As previously described the body fluid controller 116 (see FIGS. 1 and 2) is in communication with the hyperthermic heat exchanger 112 and accordingly controls the heating of a body fluid directed through the body fluid conduit 302. For instance, in one example the heating core 300 is a resistive heating core and the body fluid controller 116 controls the heating of the heating core 300 by way of the voltage applied to the heating core to thereby generate corresponding heat to raise the temperature of body fluid within the body fluid conduit 302. In another example, the heating core 300 includes a heating coil therein. For instance a medium such as heated water, steam or the like is directed through the heating core 300 to heat the body fluid present and directed through the body fluid conduit 302 of the hyperthermic heat exchanger 112.

As previously described herein, in one example the body fluid controller 116 controls the hyperthermic heat exchanger 112 to heat the body fluid provided therein from a temperature approximating the body temperature of an animal to a higher temperature, for instance a target treatment temperature, to trigger death of pathogens and pathogen infected cells in the body fluid. Referring to FIG. 2, first and second temperature sensors 202, 204 provided near to the corresponding input port 304 and output port 306 of the hyperthermic heat exchanger 112 are optionally used in combination with the body fluid controller 116 to control the heating of the heating core 300 to achieve the target treatment temperature for the body fluid within the body fluid conduit 302. For instance, an input temperature is measured with the first temperature sensor 202 and an output temperature is measured with the second temperature sensor 204. The body fluid controller 116 uses these measurements in combination with a desired target treatment temperature (determined according to the particular pathogen in question as well as a viral load of the pathogen within the body fluid) to control the hyperthermic heat exchanger 112 (e.g., the heating of the heating core 300) to heat the body fluid to the desired target treatment temperature.

In another example the hyperthermic heat exchanger 112 is operated in a non-feedback configuration, for instance the performance of the heating core 300 is known (e.g., known voltages or currents correspond to known heating core temperatures) and the body fluid controller 116 provides a specified voltage, quantity of heating medium or the like to the heating core 300. The heating core 300 correspondingly raises its temperature to a temperature corresponding to the target treatment temperature and the body fluid within the body fluid conduit 302 is heated to the target treatment temperature.

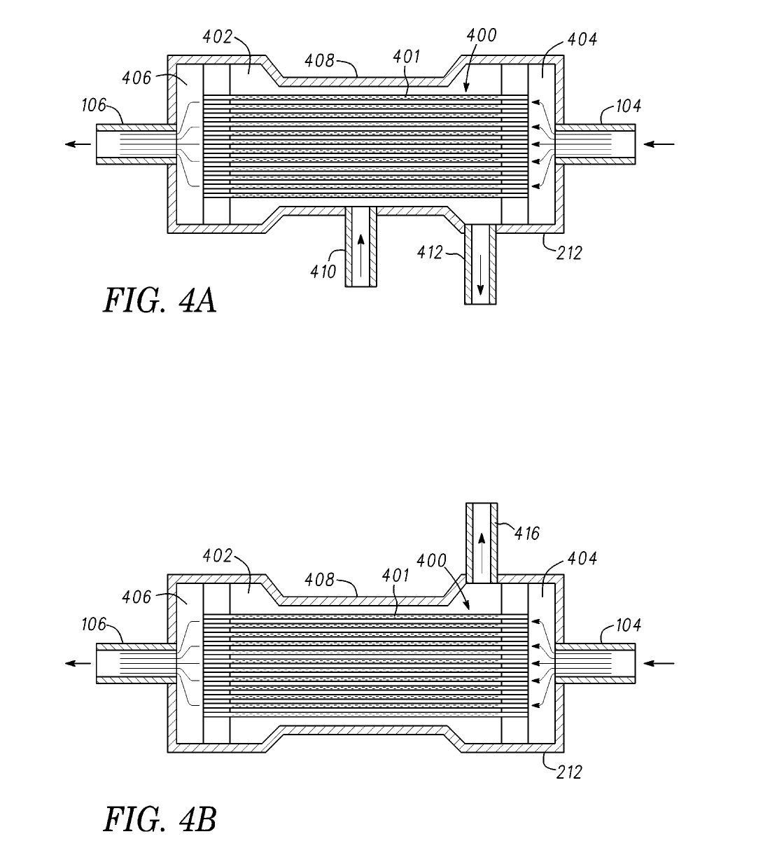

FIG. 4A shows one example of a filter 212 for use with the hyperthermic treatment system 100 shown in FIGS. 1 and 2. In one example the filter 212 is a perfusate filter including a plurality of perforations provided in one or more flow channels. Referring again to FIG. 4A, the filter 212 includes a plurality of perforated flow channels 400 including perforations 401.

The perforated flow channels 400 are provided within a reservoir jacket 408 that includes the waste removal reservoir 402. The waste removal reservoir 402 accordingly surrounds the plurality of perforated flow channels 400. As further shown in FIG. 4A, in one example the body fluid inlet 104 is in communication with an inlet manifold 404 of the filter 212. In a similar manner an outlet manifold 406 of the filter 212 is in communication with the body fluid outlet 106 (the body fluid inlet 104 and outlet 106 are both shown in FIG. 2).

As further shown in FIG. 4A and previously discussed herein, the plurality of perforated flow channels 400 are provided within the waste removal reservoir 402. An infusion port 410 and an extraction port 412 are also in communication with the waste removal reservoir 402 and accordingly the perforations 401 along the perforated flow channels 400. The infusion port 410 provides a flow of an infusion fluid, such as saline, to the waste removal reservoir 402 that washes over the perforated flow channels 400 and the perforations 401 provided there along. The flow of solution across the perforated flow channels 400 entrains waste including dead and dying pathogens and pathogen infected cells having a size and configuration sufficient (e.g., sufficiently small) to fit through the perforations 401. The infusion port 410 and the extraction port 412 provide a continuous or near continuous flow of infusion fluid while body fluid is supplied from the inlet manifold 404 through the perforated flow channels 400 and to the outlet manifold 406. The delivery of the infusion fluid to the perforated flow channels 400 continuously entrains waste from the flowing body fluid through the perforations 401 (e.g., dead and dying pathogens and pathogen infected cells) and the waste is removed through the extraction port 412. In one example the perforations 401 are provided in the perforated flow channel 400 with one or more of a specified diameter or shape ensuring passage of dead and dying pathogens and pathogen infected cells but otherwise substantially preventing the passage of other components of the body fluid, for instance red blood cells, white blood cells and the like.

Accordingly, the perforations 401 in combination with the infusion fluid provided through the infusion port 410 filter waste such as dead and dying pathogens and pathogen infected cells from the body fluid (death triggered by the hyperthermic heat exchanger 112) and thereby clean the body fluid prior to delivery to the animal through the body fluid outlet 106. Further, pathogens and pathogen infected cells are captured within the perforated flow channels 400, for instance along the walls of the channels 400 or within the perforated channels 401 to remove the pathogens and pathogen infected cells from the body fluid leaving the filter through the body fluid outlet 106.

Another example of a filter 414 is shown in FIG. 4B. At least some of the features of the filter 414 are similar to features of the filter 212 shown in FIG. 4A. For instance, the incoming body fluid is received by the filter 212 within the inlet manifold 404 from the body fluid inlet 104. The body fluid (with a reduced quantity of the pathogen and pathogen infected cells) exits the filter 212 through the outlet manifold 406 and the body fluid outlet 106.

The example of the filter 414 shown in FIG. 4B includes an extraction port 416 in communication with the waste removal reservoir 402. In operation, the pressure differential between the inlet manifold 404 and the outlet manifold 406 drives the body fluid through the perforated channels 400. As previously discussed herein, pathogens and pathogen infected cells (dead or dying) are within the body fluid and filtered out of the body fluid with the perforations 401 of the perforated channels 400. In the example shown in FIG. 4B, there is also a pressure differential between the inlet manifold 404 and the extraction port 416. The pressure differential directs a portion of the body fluid (e.g., including plasma, water and the like) through the perforations 401, into the waste removal reservoir 402 and out of the extraction port 412. The portion of the body fluid directed through the perforations 401 entrains pathogen infected cells and pathogen therein (according to the size of the perforations 401). In another example, the pathogens and pathogen infected cells are captured by the perforated channels 400, for instance the pathogens and pathogen infected cells are captured along the walls of the channels 400 or within the perforations 401. Optionally, a metering pump, syringe or the like is coupled with the extraction port 412 to enhance or generate the desired pressure differential to ensure passage of the body fluid with the entrained pathogen infected cells and pathogens into the perforations 401.

The perforated channels 400 in either of the filters 212, 414 of FIG. 4A, B optionally include one or more additives to enhance filtration. In one example, the perforated channels are coated with a lectin (e.g., a lectin media). The lectin preferentially binds to pathogens (including pathogen infected cells) and accordingly captures the pathogens along the perforated channels 400. Other components of the body fluid including, but not limited to, red and white blood cells, platelets and the like fail (or are less preferential) to bind to the lectins and thereby readily pass through the filter. Accordingly, the lectin additives enhance filtration. Eventually with saturation the lectin bound pathogens and pathogen infected cells break loose from the filter 212, 412 and are removed by way of the extraction ports 412, 416.

Lectins are generally referred to as sugar-binding proteins and are available from many biological (lectins can be purified by methods available to an artworker), as well as commercial, sources. Lectins perform recognition on the cellular and molecular level and play numerous roles in biological recognition phenomena involving cells, such as enveloping proteins in pathogens. The specificity each lectin has toward a particular carbohydrate allows one to be able to isolate a virus or viruses from a mixture (such as a mixture of cells). Various lectins include, but are not limited to, AAL, LTL, UEA I, ACL, ECL, EEL, GSL I, GSL I-B₄, Jacalin, MAL I, PNA, RCA I, RCA II, SBA, Con A, GNL, HHL, LCA, NPL, PSA, BPL, DBA, GSL I, MPL, PTL, RCA I, RCA II, SJA, SBA, VVA, WFA, DSL, GSL II, LEL, STL, WGA, MAL II, SNA, PHA-E, PHA-L and GNA. Lectins can be bound to a surface, such as a membrane and/or agarose, or to another protein by methods available to an art worker.

In another example, the hyperthermic treatment assembly 110 includes a body fluid composition sensor 213 (e.g., a blood composition sensor or blood leak sensor). The body fluid composition sensor 213 measures changes in the body fluid composition, such as the concentration of blood within the body fluid relative to other components (e.g., pathogens). The body fluid composition sensor 213 thereby provides an indication of the functionality of one or more of the preceding filter 212 (or 412) or the hyperthermic heat exchanger 112. For instance, if the blood concentration is low a higher than expected concentration of pathogen may be present and the treatment temperature may correspondingly be below a temperature configured to trigger the desired pathogen death. The body fluid controller 116 is operated to increase the treatment temperature and accordingly increase the blood concentration (by decreasing the pathogen concentration) in the manner of feedback control. In another example, a lower blood concentration may indicate the filter 212 (or 412) is not operating at a desired efficiency and service or an increased flow of saline is needed to increase the efficiency.

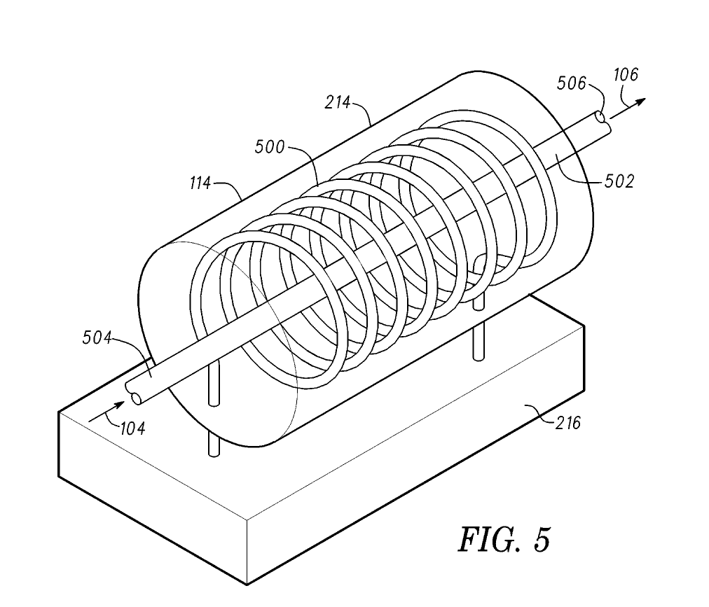

FIG. 5 shows one example of a cooling heat exchanger 114. As previously described herein in one example the cooling heat exchanger 114 is included as a component of the hyperthermic treatment assembly 110 shown in FIG. 2. Optionally, the cooling heat exchanger 114 is absent from the hyperthermic treatment assembly 110 for instance where the body fluid has a dwell time within the hyperthermic treatment assembly 110 sufficient to cool the body fluid prior to return delivery through a body fluid outlet 106 to an animal. Referring now to FIG. 5, the cooling heat exchanger 114 is shown with at least two components including a heat exchange jacket 214 and a coolant conditioner 216. In the example shown in FIG. 5 the heat exchange jacket 214 includes a cooling coil 500 extending through the heat exchange jacket 214. A body fluid conduit 502 for carrying the body fluid extends through the cooling coil 500 as shown. The body fluid conduit 502 is in communication with the body fluid inlet 104 and the body fluid outlet 106, for instance by way of corresponding input and output ports 504, 506. As further shown in FIG. 5, the cooling coil 500 and the body fluid conduit 502 are provided within the heat exchange jacket 214. In one example, the heat exchange jacket 214 includes a cavity therein to house the cooling coil 500 and the body fluid conduit 502 and bathe both of said components in a medium, such as liquid water.

The cooling coil 500 provides a flow of coolant therein such as refrigerant or chilled water to cool the body fluid within the body fluid conduit 502. For instance, the cooling coil 500 serves as a heat exchange component with the liquid within the heat exchange jacket 214. By cooling the liquid within the heating exchange jacket 214 (e.g., water) the body fluid within the body fluid conduit 502 is similarly cooled. That is to say, one or more of convective or conductive forms of heat transfer are provided between the cooling coil 500, the medium within the heat exchange jacket 214 and the body fluid conduit 502.

As further shown in FIG. 5, a coolant conditioner 216 is coupled with the cooling coil 500. The coolant conditioner 216 includes components for cooling a refrigerant or chilled water pumped through the cooling coil 500. In one example, the coolant conditioner 216 includes a refrigerant system including for chilling (and optionally changing the phase) of a refrigerant such as a compressor, a coil to facilitate heat transfer between the coolant and an exhaust medium such as air, an expansion valve, a pump and the like. In another example, the coolant conditioner 216 includes a cold water chiller including one or more features such as a miniature cooling tower or the like. As the coolant (chilled water or refrigerant) is conditioned at the coolant conditioner 216 it is pumped through the cooling coil 500 to accordingly cool the body fluid within the body fluid conduit 502 prior to delivery through the body fluid outlet 106 to return to the animal.

As previously described and shown in FIG. 2 the coolant conditioner 216 is in communication with the body fluid controller 116. In one example, the coolant conditioner 216, the second temperature sensor 204 and the third temperature sensor 206 are used in combination by the body fluid controller 116 to accordingly control the heat exchange between the body fluid and the coolant within the cooling coil 500 to thereby achieve a target return temperature prior to delivery of the body fluid through the body fluid outlet 106 for return to the animal. For example, the second temperature sensor 204 provides an input temperature of the body fluid into the cooling heat exchanger 114 and the third temperature sensor 206 provides an output temperature of the body fluid out of the cooling heat exchanger 114. The body fluid controller 116 uses these temperature inputs to control the operation of the coolant conditioner 216 (including for instance the coolant temperature, coolant flow rate and the like) to achieve a target return temperature equivalent to or slightly higher than a body temperature of the animal coupled with the hyperthermic treatment system 100 (to account for heat loss in the system 100).

As will be described herein, in one example a body fluid warmer 224 is also provided with the hyperthermic treatment system 100 to provide a final heat transfer feature for the body fluid immediately prior to delivery of the body fluid back to the animal. The body fluid warmer 224 is an optional component, and the hyperthermic heat exchanger 112 and the cooling heat exchanger 114 are used exclusively together in an example to condition the temperature of the body fluid (e.g., raise the temperature for pathogen death and lower the temperature for reintroduction).

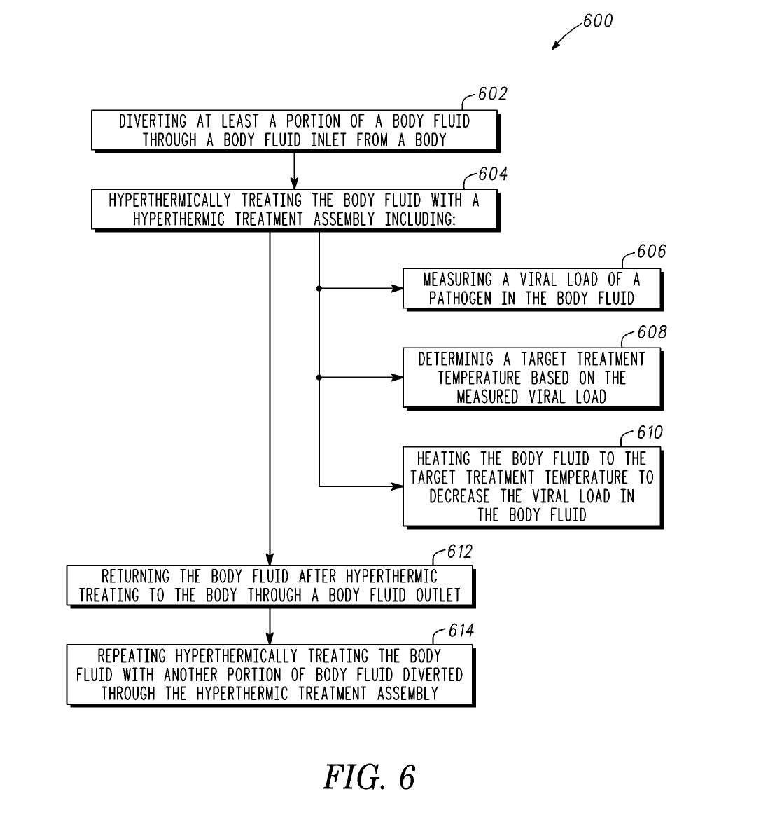

FIG. 6 shows one example of a method 600 to hyperthermically treat the body fluid of an animal, such as a human, mammal, reptile or other non-sentient animal. In describing the method 600 reference is made to one or more components, features, functions and steps previously described herein. Where convenient reference is made to the components, features, steps and the like with reference numerals. Reference numerals provided are exemplary and are not exclusive. For instance, components, features, functions, steps and the like described in the method 600 include, but are not limited to, the corresponding numbered elements provided herein other corresponding features described herein (both numbered and unnumbered) as well as their equivalents.

At 602, the method 600 includes diverting at least a portion of a body fluid through a body fluid inlet 104 from a body, for instance, a body of an animal including but not limited to a human, non-sentient animals or the like. In one example, the body fluid is delivered to a hyperthermic treatment system 100 including a hyperthermic treatment assembly 110 through a body fluid inlet 104. Optionally, the body fluid inlet 104 (as well as the body fluid outlet 106) includes a flow clamp 200 to selectively prevent and allow the flow of the body fluid into the hyperthermic treatment system 100.

At 604, the body fluid is hyperthermically treated with a hyperthermic treatment assembly 110. One example of a hyperthermic treatment assembly is shown in FIG. 2. As discussed herein, hyperthermically treating the body fluid includes raising the body fluid temperature to a target treatment temperature that will trigger cell death in the pathogen and pathogen infected cells. At 606 hyperthermically treating the body fluid includes measuring a viral load of a pathogen in the body fluid. In one example, prior to treatment of the body fluid a sample of the body fluid is taken to measure the viral load of a particular pathogen within the body fluid. In another example, the viral load of the animal is measured in an on-going manner by the hyperthermic treatment system 100. At 608, a target treatment temperature for the body fluid is determined based on the measured viral load. For instance, the body fluid controller 116 includes a database of target treatment temperatures paired with corresponding viral loads for a particular pathogen. In one example, for a particular pathogen a table is provided with increasing target treatment temperatures relative to increasing viral loads. Accordingly, with a higher measured viral load a higher target treatment temperature is identified for the hyperthermic treatment system 100.

At 610, the body fluid is heated to the target treatment temperature to decrease the viral load in the body fluid. In one example, a hyperthermic heat exchanger 112 (shown in FIG. 2) is provided inline with the hyperthermic treatment assembly 110. Optionally, the hyperthermic heat exchanger 112 is in communication with the body fluid controller 116. The body fluid controller 116 operates the hyperthermic heat exchanger 112, for instance by generating heat in a heating core 300 of the heat exchanger to correspondingly elevate the temperature of the body fluid in a body fluid conduit 302 and thereby trigger cell death in the pathogen and pathogen infected cells. In another example, heating the body fluid to the target treatment temperature includes using one or more sensors such as first and second temperature sensors 202, 204 to measure the input and output temperatures to the hyperthermic heat exchanger 112. The body fluid controller 116 uses the inputs from each of the sensors 202, 204 to correspondingly monitor and control the hyperthermic heat exchanger 112 to achieve the target treatment temperature in the body fluid flowing therethrough.

At 612, the body fluid is returned to the body after hyperthermic treatment. For instance, as shown in FIG. 2 a body fluid outlet 106 is provided in the hyperthermic treatment system 100 to facilitate the delivery of the treated body fluid back to the body of the animal. In one example, the body fluid inlet 104 is coupled with the body of the animal at a first femoral artery and the body fluid outlet is coupled at a second femoral artery.

At 614, the method 600 further includes repeating hyperthermically treating the body fluid with another portion of body fluid diverted through the hyperthermic treatment assembly 110. That is to say, in one example, the body fluid inlet 104 is coupled with the portion of the body and the body fluid outlet 106 is coupled with another portion of the body and the body fluid is cycled from the animal through the system 100 in an on-going manner. Accordingly, the hyperthermic assembly 110 is operated in a continuous fashion to facilitate the cycling of the body fluid from the animal through the treatment assembly 110 to repeatedly treat the body fluid with the hyperthermic heat exchanger 112. By conditioning the body fluid (elevating the temperature to a target treatment temperature) the viral load in the body fluid is gradually decreased as the entirety (including near entirety) of the body fluid is cycled through the hyperthermic treatment assembly 110.

Several options for the method 600 follow. In one example, repeating hyperthermically treating the body fluid includes repeating hyperthermically treating the body fluid until a target viral load is measured. For instance, in one example, the hyperthermic treatment system 100 includes an instrument in line with the hyperthermic treatment assembly 110 (e.g., coupled with the fluid port 208) configured to continuously or at intervals measure the viral load of the body fluid during treatment. Accordingly, treatment is continued and then ceased once a target viral load is reached. In another example, the viral load of the animal is determined with a standalone method, for instance, by drawing a body fluid from the animal either from the fluid port 218 or with a hypodermic syringe and testing the body fluid.

In another example, the method 600 further includes cooling the body fluid after heating to a return temperature (lower than the elevated target treatment temperature) prior to returning body fluid to the body, for instance, through the body fluid outlet 106. In an example, cooling the body fluid includes active heat exchange between the body fluid and a cooling heat exchanger, such as the cooling heat exchanger 114 shown in FIG. 2.

In still another example, heating the body fluid to the target temperature (for instance, discussed at 610) includes heating a heating core 300 of the hyperthermic heat exchanger 112 to a core temperature based on the target treatment temperature. In one example, the core temperature is higher than the target treatment temperature and accordingly accounts for heat transfer from the heating core 300 (shown in FIG. 3) to the surrounding environment and housing (the heat exchanger body 308). In still another example, heating the body fluid to the target temperature includes delivering the body fluid through a body fluid conduit 302 wrapped around the heating core 300. The heating core 300 provides conductive and convective heat transfer to the body fluid within the body fluid conduit 302 by heating the body fluid conduit 302.

In another example, the method 600 includes filtering the body fluid after heating the body fluid to the target temperature. Filtering includes removing dead or dying pathogens and dead or dying pathogen infected cells from the body fluid. One example of such a filter 212 is shown in FIG. 4A (another example is shown in FIG. 4B). The filter 212 includes a plurality of perforated flow channels 400 having perforations 401 therein. The perforated flow channels 400 receive the body fluid after heating to the target treatment temperature. The body fluid flows through the perforated flow channels 400 and a flow of infusion fluid such as saline provided through an infusion port 410 to a waste removal reservoir 402 entrains the dead and dying pathogen cells and pathogen infected cells through the perforations 401. The filtered body fluid is then delivered out of the filter 212 (e.g., to an outlet manifold 406). Optionally, the pressure differential between the inflow of fluid at the body fluid inlet 104 and an extraction port 416 draws a portion of the body fluid with entrained pathogens through the perforated flow channels 401 to the port 416.

Optionally, returning the body fluid (for instance after filtering) includes measuring a flow rate of the body fluid with the flow sensor 220. In one example, the flow rate of the body fluid is controlled according to the measured flow rate. As previously described herein, in one example, the flow sensor 220 and the flow controller 222 (an adjustable valve) are in communication with the body fluid controller 116. The body fluid controller 116 controls the flow controller 222 according to the measured flow rate through the flow sensor 220. The body fluid controller 116 in combination with the flow sensor and controller 220, 222 ensures that the inflow into the hyperthermic treatment system 100 for instance through the body fluid inlet 104 matches the outflow of the system 100 through the body fluid outlet 106.

In still another example, the method 600 further includes treating one or more chronic infectious diseases. Optionally, the hyperthermic treatment of the body fluid comprises treating one or more of, but not only, the Marberg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus and HIV.

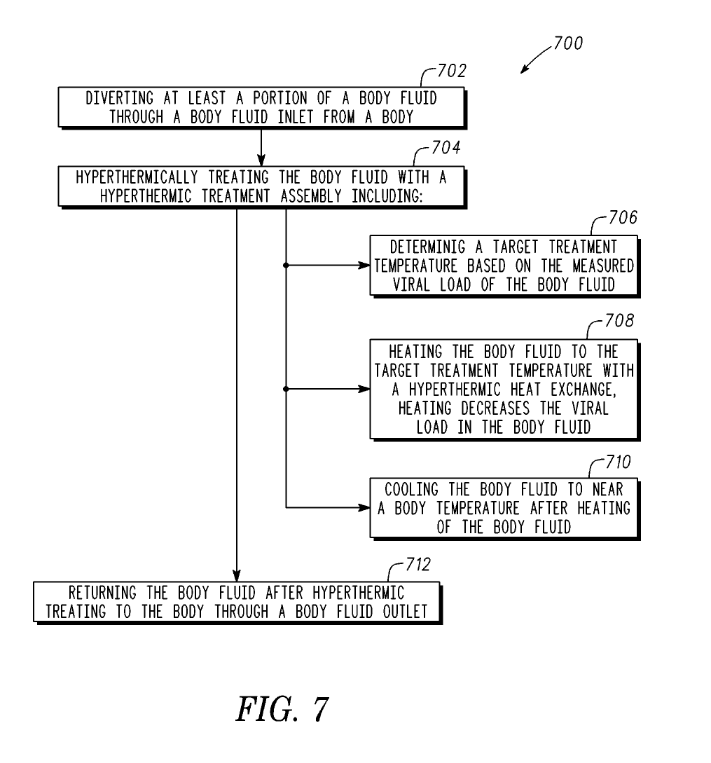

FIG. 7 shows one example of the method 700 for hyperthermically treating a body fluid of an animal. In describing the method 700 reference is made to one or more components, features, functions and steps previously described herein. Where convenient reference is made to the components, features, steps and the like with reference numerals. Reference numerals provided are exemplary and are not exclusive. For instance, components, features, functions, steps and the like described in the method 700 include but are not limited to the corresponding numbered elements provided herein, other corresponding features described herein both numbered and unnumbered as well as their equivalents.

At 702, the method 700 includes diverting at least a portion of a body fluid through a body fluid inlet 104 from a body. At 704, the body fluid is hyperthermically treated with the hyperthermic treatment assembly 110. In one example, the hyperthermic treatment assembly 110 shown in FIG. 2. As discussed herein, hyperthermically treating the body fluid includes elevating the temperature of the body fluid to a target treatment temperature that triggers cell death of the pathogen and pathogen infected cells. Hyperthermically treating the body fluid includes at 706 determining the target treatment temperature based on a measured viral load of the body fluid. As previously described herein, in one example a testing procedure is conducted for the animal to determine the viral load of a particular pathogen in the animal. The target treatment temperature is determined based on this viral load and the particular pathogen detected. For instance, the body fluid controller 116 includes a database including a plurality of target treatment temperatures associated with particular viral loads of a specified pathogen. Optionally, the database includes a catalogue of pathogens with corresponding viral loads and associated target treatment temperatures for each of those viral loads.

Hyperthermically treating the body fluid further includes at 708 heating the body fluid to the target treatment temperature with the hyperthermic heating exchanger 112. For instance, a heating core 300 is heated to accordingly heat a body fluid conduit 302 of the hyperthermic heat exchanger 112. Heating the body fluid decreases the viral load in the body based on the heat applied to the body fluid.

At 710, the body fluid is cooled to near a body temperature (matches the body temperature or is near to the body temperature) after heating of the body fluid and prior to returning the body fluid to the body, for instance through a body fluid outlet 106. As previously described herein, in one example, cooling the body fluid includes active heat exchange, for instance conducted by a cooling heat exchanger 104 provided downstream from the hyperthermic heat exchanger 112.

At 712, the method 700 includes returning the body fluid after hyperthermic treatment to the body through a body fluid outlet 106. As discussed herein, the returning body fluid has a decreased viral load as a function of the hyperthermic treatment, for instance with the hyperthermic heat exchanger 112. Additionally, the returning body fluid is provided to the body at a return temperature near to body temperature and lower than the target treatment temperature achieved with the hyperthermic heat exchanger 112.

Several options for the method 700 follow. In one example, the method 700 includes repeating hyperthermically treated the body fluid with another portion of body fluid diverted through the hyperthermic treatment assembly 110. As previously described herein, in one example, the body fluid inlet 104 is coupled with a femoral artery and the body fluid outlet 106 is coupled with an opposed femoral artery. Diverting at least a portion of the body fluid through a body fluid inlet from the body includes continuously diverting portions of the body fluid therethrough. Accordingly, the body fluid is cycled in a continuous or near continuous fashion through the hyperthermic treatment assembly 110 to condition all or a large portion of the body fluid of the animal to accordingly decrease the viral load throughout the volume of the body fluid.

In another example, hyperthermically treating the body fluid, for instance cooling the body fluid, includes measuring a first output temperature of the body fluid near an output of the hyperthermic heat exchanger 112 for instance the second temperature sensor 204 shown in FIG. 2 and measuring an output of the cooling heat exchanger 104 for instance at the third temperature sensor 206 also shown in FIG. 2. Cooling the body fluid to near body temperature after heating (with the hyperthermic heat exchanger 112) includes cooling according to the measured first and second output temperatures (measured at the respective sensors 204, 206). That is to say, in one example a controller such as the body fluid controller 116 of the hyperthermic treatment assembly 110 is used in cooperation with the second and third temperature sensors 204, 206 to control active heat exchange at the cooling heat exchanger 114 to thereby lower the temperature of the body fluid to a return temperature near that of the body.

In a similar manner, hyperthermically treating the body fluid (heating) includes measuring a first input temperature of the body fluid near an input of the hyperthermic heat exchanger 112, for instance with a first temperature sensor 202, and measuring a first output temperature of the body fluid near an output of the hyperthermic heat exchanger, for instance with the second temperature sensor 204. Heating the body fluid to the target treatment temperature includes heating to the target treatment temperature according to the measured first input and first output temperatures. That is to say, in another example the body fluid controller 116 uses the inputs from the first and second temperature sensors 202, 204 corresponding to input and output temperatures for the hyperthermic heat exchanger 112 to control the heating of the hyperthermic heat exchanger 102 (e.g., a heating core 300) to achieve the target treatment temperature accurately and precisely to ensure a decrease of the viral load to the desired degree.

In another example, the method 700 includes filtering the body fluid after heating the body fluid to the target treatment temperature. Filtering includes removing dead or dying pathogens and dead or dying pathogen infected cells from the body fluid. One example of such a filter 212 is shown in FIG. 4A and includes a plurality of perforated flow channels 400 and a cross-flow of infusion fluid provided by an infusion port 410 and drawn out of the filter 212 through an extraction port 412. The infusion fluid draws dead or dying pathogen cells and pathogen infected cells through the perforations 401 and entrains the same within the infusion fluid for eventual extraction through the extraction port 412. Optionally, the filter 414 shown in FIG. 4B is used.

In another example, the method 700 includes measuring a flow rate of the body fluid with the flow sensor 220 shown in FIG. 2 and controlling the flow rate of the body fluid according to the measured flow rate for instance with a flow controller 222 in communication with the body fluid controller 116.

In still another example the method 700 includes treating one or more chronic infectious diseases. Chronic infectious diseases treated with the hyperthermic treatment of body fluid (at 704) include, but are not limited to, Marberg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus and HIV.

One prophetic example of a therapy scheme for hyperthermic treatment of a body fluid infected with a pathogen is provided below. The prophetic example is provided as an exemplary schema and accordingly the target treatment temperatures provided for differing measured viral loads will vary according to the pathogen treated. In the prophetic example, the pathogen includes one or more of Crimean Congo hemorrhagic fever, Dengue fever, Dengue fever-febrile, Dengue fever-defevrescent, Dengue hemorrhagic fever, Dengue hemorrhagic fever-febrile, Dengue hemorrhagic fever-defevrsescent, Ebola, Hepatitis C virus, HIV, Lassa virus, Rift Valley fever or Sin Nombre. The exemplary therapy scheme for hyperthermic treatment as described herein includes a graduated series of treatment temperatures that vary according to measured viral loads of a body fluid. The exemplary therapy scheme is provided below in table form.

| Measured Viral Load (in units of | | Body Fluid Target |
|---|---|---|
| pathogen copies per milliliter of body | | Treatment Temperature |
| fluid) | | (in degrees Fahrenheit) |
| 50 | copies/ml | 102 degrees F. |
| 10k-30k | copies/ml | 105 degrees F. |
| 100k | copies/ml | 110 degrees F. |

As shown, the target treatment temperature (and the heating provided by the hyperthermic heat exchanger) increases with higher measured viral loads. In one example, the hyperthermic treatment assembly 100 is continually operated and the target treatment temperature regulated (up or down) according to the measurement of the viral load. For instance, as the measured viral load decreases the hyperthermic heat exchanger 112 (controlled by the body fluid controller 116) gradually decreases the target treatment temperature to the corresponding temperature stored for a particular therapy scheme.

In a similar manner to the prophetic example provided above the hyperthermic treatment assembly 110 may include or receive one or more therapy schemes for each of a variety of pathogens, each scheme with differing target treatment temperatures associated with measured viral loads. Example pathogens include, but are not limited to, Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Malaria or HIV. The temperature of the body fluid of the subject (e.g., blood) is raised with the methods and devices described herein to a temperature (including a range of temperatures) and a duration of elevated temperature sufficient to reduce the viral load of the subject by 40 percent or more. Optionally, before, after or during hyperthermic treatment, the subject is treated with a follow up therapy of immunotherapy to further treat the subject for the pathogen.

### Various Notes & Examples

Example 1 can include subject matter, such as can include a method of hyperthermic treatment of body fluid of a human patient comprising: diverting at least a portion of a body fluid through a body fluid inlet from a body; hyperthermically treating the body fluid with a hyperthermic treatment assembly including: measuring a viral load of a pathogen in the body fluid, determining a target treatment temperature based on the measured viral load, and heating the body fluid to the target treatment temperature to decrease the viral load in the body fluid; returning the body fluid after hyperthermic treating to the body through a body fluid outlet; and repeating hyperthermically treating the body fluid with another portion of body fluid diverted through the hyperthermic treatment assembly.

Example 2 can include, or can optionally be combined with the subject matter of Example 1, to optionally include wherein repeating hyperthermically treating the body fluid includes repeating hyperthermically treating the body fluid until a target viral load is measured.

Example 3 can include, or can optionally be combined with the subject matter of one or any combination of Examples 1 or 2 to optionally include cooling the body fluid after heating to a return temperature prior to returning the body fluid to the body.

Example 4 can include, or can optionally be combined with the subject matter of one or any combination of Examples 1-3 to optionally include wherein the cooling the body fluid includes active heat exchange between the body fluid and a cooling heat exchanger.

Example 5 can include, or can optionally be combined with the subject matter of one or any combination of Examples 1-4 to optionally include wherein heating the body fluid to the target temperature includes: heating a heating core to a core temperature based on the target treatment temperature, and delivering the body fluid through a body fluid conduit wrapped around the heating core and heating the body fluid to the target treatment temperature with the heating core.

Example 6 can include, or can optionally be combined with the subject matter of Examples 1-5 to optionally include filtering the body fluid after heating the body fluid to the target treatment temperature, filtering including removing dead or dying pathogens and dead or dying pathogen infected cells from the body fluid.

Example 7 can include, or can optionally be combined with the subject matter of Examples 1-6 to optionally include filtering the body fluid after heating the body fluid to the target treatment temperature, filtering including capturing dead or dying pathogens and dead or dying pathogen infected cells with a lectin media coupled along a filter.

Example 8 can include, or can optionally be combined with the subject matter of Examples 1-7 to optionally include wherein returning the body fluid after hyperthermic treating includes: measuring a flow rate of the body fluid, and controlling the flow rate of the body fluid according to the measured flow rate.

Example 9 can include, or can optionally be combined with the subject matter of Examples 1-8 to optionally include wherein diverting at least the portion of the body fluid includes diverting at least the portion of the body fluid from a first femoral artery, and returning the body fluid after hyperthermic treating includes returning the body fluid to a second femoral artery.

Example 10 can include, or can optionally be combined with the subject matter of Examples 1-9 to optionally include wherein the hyperthermic treatment of body fluid comprises treating one or more chronic infectious diseases.

Example 11 can include, or can optionally be combined with the subject matter of Examples 1-10 to optionally include wherein the hyperthermic treatment of body fluid comprises treating at least one of Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Yellow Fever and HIV.

Example 12 can include, or can optionally be combined with the subject matter of Examples 1-11 to optionally include administering immunotherapy treatment to the body fluid between diverting and returning of the body fluid.

Example 13 can include, or can optionally be combined with the subject matter of Examples 1-12 to optionally include a method of hyperthermic treatment of body fluid of a human patient comprising: diverting at least a portion of a body fluid through a body fluid inlet from a body; hyperthermically treating the body fluid with a hyperthermic treatment assembly including: determining a target treatment temperature based on a measured viral load of the body fluid, heating the body fluid to the target treatment temperature with a hyperthermic heat exchanger, heating decreases the viral load in the body fluid, cooling the body fluid to near a body temperature after heating of the body fluid; and returning the body fluid after hyperthermic treating to the body through a body fluid outlet.

Example 14 can include, or can optionally be combined with the subject matter of Examples 1-13 to optionally include repeating hyperthermically treating the body fluid with another portion of body fluid diverted through the hyperthermic treatment assembly. Example 15 can include, or can optionally be combined with the subject matter of

Examples 1-14 to optionally include wherein hyperthermically treating the body fluid includes: measuring a first output temperature of the body fluid near an output of the hyperthermic heat exchanger and measuring a second output temperature of the body fluid near an output of a cooling heat exchanger downstream from the hyperthermic heat exchanger, and cooling the body fluid to near body temperature after heating includes cooling according to the measured first and second output temperatures.

Example 16 can include, or can optionally be combined with the subject matter of Examples 1-15 to optionally include wherein hyperthermically treating the body fluid includes: measuring a first input temperature of the body fluid near an input of the hyperthermic heat exchanger and measuring a first output temperature of the body fluid near an output of the hyperthermic heat exchanger, and heating the body fluid to the target treatment temperature includes heating to the target treatment temperature according to the measured first input and first output temperatures.

Example 17 can include, or can optionally be combined with the subject matter of Examples 1-16 to optionally include wherein the cooling the body fluid to near body temperature includes active heat exchange between the body fluid and a cooling heat exchanger.

Example 18 can include, or can optionally be combined with the subject matter of Examples 1-17 to optionally include wherein heating the body fluid to the target temperature includes: heating a heating core to a core temperature based on the target treatment temperature, and delivering the body fluid through a body fluid conduit wrapped around the heating core and heating the body fluid to the target treatment temperature with the heating core.

Example 19 can include, or can optionally be combined with the subject matter of Examples 1-18 to optionally include filtering the body fluid after heating the body fluid to the target treatment temperature, filtering including removing dead or dying pathogens and dead or dying pathogen infected cells from the body fluid.

Example 20 can include, or can optionally be combined with the subject matter of Examples 1-19 to optionally include filtering the body fluid after heating the body fluid to the target temperature, filtering including capturing dead or dying pathogens and dead or dying pathogen infected cells with a lectin media coupled along a filter.

Example 21 can include, or can optionally be combined with the subject matter of Examples 1-20 to optionally include wherein returning the body fluid after hyperthermic treating includes: measuring a flow rate of the body fluid, and controlling the flow rate of the body fluid according the measured flow rate.

Example 22 can include, or can optionally be combined with the subject matter of Examples 1-21 to optionally include wherein the hyperthermic treatment of body fluid comprises treating one or more chronic infectious diseases.

Example 23 can include, or can optionally be combined with the subject matter of Examples 1-22 to optionally include wherein the hyperthermic treatment of body fluid comprises treating at least one of Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Yellow Fever, and HIV.

Example 24 can include, or can optionally be combined with the subject matter of Examples 1-23 to optionally include a hyperthermic treatment system for use with body fluids of a human patient comprising: a body fluid inlet; a body fluid outlet; a body fluid pump in communication with the body fluid inlet and outlet, the body fluid pump moves body fluid through the hyperthermic treatment system; and a hyperthermic treatment assembly in communication with the body fluid inlet and outlet, the hyperthermic treatment circuit includes: a hyperthermic heat exchanger interposed between the body fluid inlet and outlet, the hyperthermic heat exchanger heats the body fluid to a target treatment temperature, a cooling heat exchanger interposed between the hyperthermic heat exchanger and the body fluid outlet, the cooling heat exchanger cools the body fluid after heating by the hyperthermic heat exchanger, and a body fluid controller coupled with at least the hyperthermic heat exchanger, the body fluid controller controls the hyperthermic heat exchanger to heat the body fluid to the target treatment temperature.

Example 25 can include, or can optionally be combined with the subject matter of Examples 1-24 to optionally include wherein the body fluid controller is in communication with the cooling heat exchanger, and the body fluid controller controls the cooling heat exchanger to cool the body fluid to near a body temperature.

Example 26 can include, or can optionally be combined with the subject matter of Examples 1-25 to optionally include wherein the controller includes a viral load temperature module, the viral load temperature module includes a database of a plurality of viral load values and a plurality of target treatment temperatures, each of the viral load values is associated with a corresponding target treatment temperature of the plurality of target treatment temperatures.

Example 27 can include, or can optionally be combined with the subject matter of Examples 1-26 to optionally include wherein the hyperthermic treatment assembly includes: a first temperature sensor upstream from the hyperthermic heat exchanger, a second temperature sensor downstream from the hyperthermic heat exchanger, and wherein each of the first and second temperature sensors is coupled with the body fluid controller.

Example 28 can include, or can optionally be combined with the subject matter of Examples 1-27 to optionally include wherein the second temperature sensor is upstream from the cooling heat exchanger, and the hyperthermic treatment assembly includes a third temperature sensor downstream from the cooling heat exchanger, and the third temperature sensor is coupled with the body fluid controller.

Example 29 can include, or can optionally be combined with the subject matter of Examples 1-28 to optionally include a perfusate filter downstream from the hyperthermic heat exchanger, the perfusate filter includes: a plurality of perforated flow channels, and a waste removal reservoir around the plurality of perforated flow channels, the plurality of perforated flow channels are in communication with the waste removal reservoir through perforations in the perforated flow channels.

Example 30 can include, or can optionally be combined with the subject matter of Examples 1-29 to optionally include wherein the plurality of perforated flow channels include a lectin media coupled along flow channel surfaces.

Example 31 can include, or can optionally be combined with the subject matter of Examples 1-30 to optionally include wherein the hyperthermic heat exchanger includes: a heating core, and a body fluid conduit in communication with the body fluid inlet and outlet, wherein the body fluid conduit wraps around the heating core.

Example 32 can include, or can optionally be combined with the subject matter of Examples 1-31 to optionally include wherein the cooling heat exchanger includes: a cooling coil extending within a cooling jacket, and a body fluid conduit extending through the cooling coil and within the cooling jacket.

Example 33 can include, or can optionally be combined with the subject matter of Examples 1-32 to optionally include a flow sensor downstream from at least the hyperthermic heat exchanger, a flow controller adjacent to the flow sensor, and wherein the flow sensor and the flow controller are coupled with the body fluid controller, and the body fluid controller adjusts a flow controller output according to measurements of a body fluid flow from the flow sensor.

Each of these non-limiting examples can stand on its own, or can be combined in any permutation or combination with any one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions. Embodiments defined by each of these transition terms are within the scope of this invention.

Moreover, in the following claims, the terms “first,” “second,” and “third,” etc. are used merely as labels, and are not intended to impose numerical requirements on their objects.

All numerical designations, e.g., pH, temperature, time, concentration, and molecular weight, including ranges, are approximations which are varied (+) or (−) by increments of 0.1. It is to be understood, although not always explicitly stated, that the reagents described herein are merely exemplary and that equivalents of such are known in the art.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method of hyperthermic treatment of body fluid of a human patient comprising:
diverting at least a portion of a body fluid through a body fluid inlet from a body;
hyperthermically treating the body fluid with a hyperthermic treatment assembly including:
measuring a viral load of a pathogen in the body fluid,
determining a target treatment temperature based on the measured viral load, and
heating the body fluid to the target treatment temperature to decrease the viral load in the body fluid;
returning the body fluid after hyperthermic treating to the body through a body fluid outlet; and
repeating hyperthermically treating the body fluid with another portion of body fluid diverted through the hyperthermic treatment assembly.

2. The method of claim 1, wherein repeating hyperthermically treating the body fluid includes repeating hyperthermically treating the body fluid until a target viral load is measured.

3. The method of claim 1 comprising cooling the body fluid after heating to a return temperature prior to returning the body fluid to the body.

4. The method of claim 3, wherein the cooling the body fluid includes active heat exchange between the body fluid and a cooling heat exchanger.

5. The method of claim 1, wherein heating the body fluid to the target temperature includes:
heating a heating core to a core temperature based on the target treatment temperature, and
delivering the body fluid through a body fluid conduit wrapped around the heating core and heating the body fluid to the target treatment temperature with the heating core.

6. The method of claim 1 comprising filtering the body fluid after heating the body fluid to the target treatment temperature, filtering including removing dead or dying pathogens and dead or dying pathogen infected cells from the body fluid.

7. The method of claim 1 comprising filtering the body fluid after heating the body fluid to the target treatment temperature, filtering including capturing dead or dying pathogens and dead or dying pathogen infected cells with a lectin media coupled along a filter.

8. The method of claim 1, wherein returning the body fluid after hyperthermic treating includes:
measuring a flow rate of the body fluid, and
controlling the flow rate of the body fluid according to the measured flow rate.

9. The method of claim 1, wherein diverting at least the portion of the body fluid includes diverting at least the portion of the body fluid from a first femoral artery, and returning the body fluid after hyperthermic treating includes returning the body fluid to a second femoral artery.

10. The method of claim 1, wherein the hyperthermic treatment of body fluid comprises treating one or more chronic infectious diseases.

11. The method of claim 1, wherein the hyperthermic treatment of body fluid comprises treating at least one of Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Yellow Fever and HIV.

12. The method of claim 1 comprising administering immunotherapy treatment to the body fluid between diverting and returning of the body fluid.

13. A method of hyperthermic treatment of body fluid of a human patient comprising:
diverting at least a portion of a body fluid through a body fluid inlet from a body;
hyperthermically treating the body fluid with a hyperthermic treatment assembly including:
determining a target treatment temperature based on a measured viral load of the body fluid,
heating the body fluid to the target treatment temperature with a hyperthermic heat exchanger, heating decreases the viral load in the body fluid,
cooling the body fluid to near a body temperature after heating of the body fluid; and
returning the body fluid after hyperthermic treating to the body through a body fluid outlet.

14. The method of claim 13 comprising repeating hyperthermically treating the body fluid with another portion of body fluid diverted through the hyperthermic treatment assembly.

15. The method of claim 13, wherein hyperthermically treating the body fluid includes:
measuring a first output temperature of the body fluid near an output of the hyperthermic heat exchanger and measuring a second output temperature of the body fluid near an output of a cooling heat exchanger downstream from the hyperthermic heat exchanger, and
cooling the body fluid to near body temperature after heating includes cooling according to the measured first and second output temperatures.

16. The method of claim 13, wherein hyperthermically treating the body fluid includes:
measuring a first input temperature of the body fluid near an input of the hyperthermic heat exchanger and measuring a first output temperature of the body fluid near an output of the hyperthermic heat exchanger, and
heating the body fluid to the target treatment temperature includes heating to the target treatment temperature according to the measured first input and first output temperatures.

17. The method of claim 13, wherein the cooling the body fluid to near body temperature includes active heat exchange between the body fluid and a cooling heat exchanger.

18. The method of claim 13, wherein heating the body fluid to the target temperature includes:
heating a heating core to a core temperature based on the target treatment temperature, and
delivering the body fluid through a body fluid conduit wrapped around the heating core and heating the body fluid to the target treatment temperature with the heating core.

19. The method of claim 13 comprising filtering the body fluid after heating the body fluid to the target treatment temperature, filtering including removing dead or dying pathogens and dead or dying pathogen infected cells from the body fluid.

20. The method of claim 13 comprising filtering the body fluid after heating the body fluid to the target temperature, filtering including capturing dead or dying pathogens and dead or dying pathogen infected cells with a lectin media coupled along a filter.

21. The method of claim 13, wherein returning the body fluid after hyperthermic treating includes:
measuring a flow rate of the body fluid, and
controlling the flow rate of the body fluid according the measured flow rate.

22. The method of claim 13, wherein the hyperthermic treatment of body fluid comprises treating one or more chronic infectious diseases.

23. The method of claim 13, wherein the hyperthermic treatment of body fluid comprises treating at least one of Marburg virus, Ebola, Hantavirus, H5N1 strain of bird flu, Lassa virus, Junin virus, Dengue fever, Crimea-Congo fever virus, Bolivian hemorrhagic fever, Kyasanur Forestirus (KFD) virus, Yellow Fever, and HIV.

24. A hyperthermic treatment system for use with body fluids of a human patient comprising:
a body fluid inlet;
a body fluid outlet;
a body fluid pump in communication with the body fluid inlet and outlet, the body fluid pump moves body fluid through the hyperthermic treatment system; and
a hyperthermic treatment assembly in communication with the body fluid inlet and outlet, the hyperthermic treatment circuit includes:
a hyperthermic heat exchanger interposed between the body fluid inlet and outlet, the hyperthermic heat exchanger heats the body fluid to a target treatment temperature,
a cooling heat exchanger interposed between the hyperthermic heat exchanger and the body fluid outlet, the cooling heat exchanger cools the body fluid after heating by the hyperthermic heat exchanger, and
a body fluid controller coupled with at least the hyperthermic heat exchanger, the body fluid controller controls the hyperthermic heat exchanger to heat the body fluid to the target treatment temperature.

25. The hyperthermic treatment system of claim 24, wherein the body fluid controller is in communication with the cooling heat exchanger, and the body fluid controller controls the cooling heat exchanger to cool the body fluid to near a body temperature.

26. The hyperthermic treatment system of claim 24, wherein the controller includes a viral load temperature module, the viral load temperature module includes a database of a plurality of viral load values and a plurality of target treatment temperatures, each of the viral load values is associated with a corresponding target treatment temperature of the plurality of target treatment temperatures.

27. The hyperthermic treatment system of claim 24, wherein the hyperthermic treatment assembly includes:
a first temperature sensor upstream from the hyperthermic heat exchanger,
a second temperature sensor downstream from the hyperthermic heat exchanger, and
wherein each of the first and second temperature sensors is coupled with the body fluid controller.

28. The hyperthermic treatment system of claim 27, wherein the second temperature sensor is upstream from the cooling heat exchanger, and the hyperthermic treatment assembly includes a third temperature sensor downstream from the cooling heat exchanger, and the third temperature sensor is coupled with the body fluid controller.

29. The hyperthermic treatment system of claim 24 comprising a perfusate filter downstream from the hyperthermic heat exchanger, the perfusate filter includes:
a plurality of perforated flow channels, and
a waste removal reservoir around the plurality of perforated flow channels, the plurality of perforated flow channels are in communication with the waste removal reservoir through perforations in the perforated flow channels.

30. The hyperthermic treatment system of claim 29, wherein the plurality of perforated flow channels include a lectin media coupled along flow channel surfaces.

31. The hyperthermic treatment system of claim 24, wherein the hyperthermic heat exchanger includes:
a heating core, and
a body fluid conduit in communication with the body fluid inlet and outlet, wherein the body fluid conduit wraps around the heating core.

32. The hyperthermic treatment system of claim 24, wherein the cooling heat exchanger includes:
a cooling coil extending within a cooling jacket, and
a body fluid conduit extending through the cooling coil and within the cooling jacket.

33. The hyperthermic treatment system of claim 24 comprising:
a flow sensor downstream from at least the hyperthermic heat exchanger,
a flow controller adjacent to the flow sensor, and
wherein the flow sensor and the flow controller are coupled with the body fluid controller, and the body fluid controller adjusts a flow controller output according to measurements of a body fluid flow from the flow sensor.
